(19) United States
(12) Patent Application Publication
(10) Pub. No.: US 2026/0290555 A1
(43) Pub. Date: Sep. 24, 2026

(54) IMAGING PROTOCOL REPOSITORY FOR AUTOMATIC PROTOCOL UPDATE RECOMMENDATIONS AND A VENDOR-AGNOSTIC SYSTEM AND METHOD FOR RAISING PROTOCOL-RELATED ALERTS

(71) Applicant: KONINKLIJKE PHILIPS N.V., EINDHOVEN (NL)

(72) Inventors: JESSICA WANG, CAMBRIDGE, MA (US); EKIN KOKER, CAMBRIDGE, MA (US); XINYU WANG, HAMBURG (DE); RANJITH NAVEEN TELLIS, TEWKSBURY, MA (US); OLGA STAROBINETS, NEWTON, MA (US); SIVA CHAITANYA CHADUVULA, MALDEN, MA (US); FALK UHLEMANN, NORDERSTEDT (DE)

(21) Appl. No.: 19/168,856

(22) PCT Filed: Mar. 25, 2024
(86) PCT No.: PCT/EP2024/057930

§ 371 Date: Sep. 25, 2025

Related U.S. Application Data
(60) Provisional application No. 63/455,637, filed on Mar. 30, 2023.

(51) Int. Cl. G16H 30/20; G16H 30/40; G16H 40/63; G16H 40/67; G16H 70/20

(52) U.S. Cl. CPC G16H 30/20; G16H 30/40; G16H 40/63; G16H 40/67; G16H 70/20

(57) ABSTRACT

Medical literature is processed to extract suggested updates to an imaging protocols repository of a medical facility and adding the suggested updates to a protocol update suggestions repository. During a medical imaging procedure, a medical imaging protocol is identified from the imaging protocols repository being used in the medical imaging procedure. A protocol update suggestion for the identified medical imaging protocol is retrieved from the protocol update suggestions repository, and displayed on a display device observable by an operator performing the medical imaging procedure. During the medical imaging procedure, examination monitoring information is transmitted to a remote user. From the examination monitoring information, an imaging protocol being employed during the medical imaging examination is identified. The examination monitoring information and the identified imaging protocol are compared to detect one or more deviations from the identified imaging protocol, and an indication of the detected one or more deviations is displayed.

The following relates generally to the imaging arts, remote imaging assistance arts, remote imaging examination monitoring arts, and related arts.

### BACKGROUND

Medical imaging, such as computed tomography (CT) imaging, magnetic resonance imaging (MRI), positron emission tomography (PET) imaging, fluoroscopy imaging, and so forth, is a valuable component of providing medical care, and is used in a wide range of medical fields, such as cardiology, oncology, neurology, orthopedics, to name a few. The operator of the medical imaging device used to acquire the medical images is typically a trained technologist, while interpretation of the medical images is often handled by a medical specialist such as a radiologist.

CT and MRI are powerful imaging modalities used by clinicians and researchers to non-invasively visualize human anatomy and pathology in great detail. Image contrast depends primarily on the parameters and sequences used to acquire the images, and CT and MRI exams are categorized by protocol type depending on which organ/body part they have originally been developed and optimized for. CT protocols vary in scan parameters and MRI protocols typically include numerous distinct sequences to obtain different anatomic and pathologic information from images.

Researchers and radiologists are constantly working to optimize parameters, improve existing sequences, and establish new imaging techniques for faster scans of higher image quality and lower radiation doses when applicable. However, these changes are often not reflected in protocols actually used by imaging centers. Standardization of CT and MRI protocols has been recommended by researchers and clinicians to ensure consistency across healthcare institutions, but most imaging centers develop and maintain their own imaging protocols.

CT and MRI protocols and guidelines are not standardized and often vary significantly between healthcare institutions depending on several factors including level of expertise, hardware and software limitations, and resource availability. For example, a major academic hospital may have the capability to update existing protocols with the latest advancements in imaging research, whereas a smaller community health center may lack the expertise, time, and money to do so. However, this can lead to major disparities in the quality of care that patients receive; updated scan protocols may generate images that are more accurate and show greater detail in a shorter amount of time than older protocols. As a result, patients who have access to imaging centers that routinely update their protocols may receive higher quality medical imaging examinations. Often, these larger imaging centers are in major urban cities that may not be accessible to all patients seeking care; patients from vulnerable, low socioeconomic status, minority, and rural groups are more likely to be medically underserved due to barriers to accessing healthcare including being uninsured, lack of affordable transportation, and limited appointment times that are limited to business hours, to name a few.

Typically, radiology departments source protocol updates and make adjustments manually, which is time-intensive, inefficient, and takes away from time for tasks of higher value. In addition, research studies are often tested in controlled environments (limited set of scanners, patient groups, etc.) and the findings constantly evolve. This poses a challenge for technologists or imaging centers to use a research-developed protocol confidently in their commercial setting.

A remote operations command center (ROCC) can be used to support imaging centers. The ROCC infrastructure provides bay cameras and control display/console mirroring to provide a remote expert (e.g. a senior imaging technologist or radiologist, or a servicing technician) with situational awareness of an imaging examination, and the ROCC further provides telephonic or video call capability for the remote expert to advise a local operator actually performing the imaging examination. In the ROCC setting, remote experts have access to a radiology information system (RIS) through which they can examine the imaging order corresponding to the upcoming or ongoing exam and get a sense of imaging procedures needed for the patient. When offering support to a local operator through a ROCC platform, a remote expert needs similar details on imaging procedures such as body part being scanned, protocol used, patient positioning, scan progress etc. used by the local operator. Such information helps remote experts not only to verify/validate the exam procedure with the RIS, but also assist them in deciding if any intervention is needed. There is little uniformity on the imaging procedure details across different vendors. In other words, visibility of imaging procedure details such as protocol name, body part being scanned, are not always readily available on these screens.

Protocol adherence is important to ensure image quality and patient safety. Remote technologists should ensure that the local operators select the protocols as per pre-defined policies of their imaging center. As remote technologists support scanners from different vendors, it is important to provide information on the current exam to the remote technologist in a uniform and consistent manner. However, information availability related to an ongoing exam is not uniform or available or displayed at all times across vendors. This kind of variability poses challenges in extracting the details from the console screen in a reliable generic way.

The following discloses certain improvements to overcome these problems and others.

### SUMMARY

In one aspect, a non-transitory computer readable medium stores instructions executable by at least one electronic processor to perform a method for assisting a medical imaging examination of a patient. The method includes: processing medical literature to extract suggested updates to an imaging protocols repository of a medical facility and adding the suggested updates to a protocol update suggestions repository; during a medical imaging procedure, identifying a medical imaging protocol from the imaging protocols repository being used in the medical imaging procedure; retrieving a protocol update suggestion for the identified medical imaging protocol from the protocol update suggestions repository; and displaying the retrieved protocol update suggestion on a display device observable by an operator performing the medical imaging procedure.

In another aspect, an apparatus is disclosed for assisting a medical imaging examination of a patient performed using a medical imaging device controlled by a controller having a display. The apparatus includes a screen mirroring connection configured to perform screen mirroring of the display of the controller of the medical imaging device; and at least one electronic processor programmed to perform a method for assisting the medical imaging examination of the patient. The method includes: receiving examination monitoring information related to the medical imaging examination of the patient and transmitting the examination monitoring information to a remote user, the examination monitoring information including at least a screen mirroring data stream of the display of the controller of the medical imaging device; identifying, from the examination monitoring information, an imaging protocol being employed during the medical imaging examination; comparing the examination monitoring information and the identified imaging protocol to detect one or more deviations from the identified imaging protocol; and displaying, on a display device, an indication of the detected one or more deviations.

In another aspect, a method for assisting a medical imaging examination of a patient includes: receiving examination monitoring information related to the medical imaging procedure and transmitting the examination monitoring information to a remote user, the examination monitoring information including at least a screen mirroring data stream of a controller of a medical imaging device used to perform the medical imaging examination of the patient; processing medical literature to extract suggested updates to an imaging protocols repository of a medical facility and adding the suggested updates to a protocol update suggestions repository based on the received examination monitoring information; during a medical imaging procedure, identifying a medical imaging protocol from the imaging protocols repository being used in the medical imaging procedure; retrieving a protocol update suggestion for the identified medical imaging protocol from the protocol update suggestions repository; and displaying the retrieved protocol update suggestion on a display device observable by an operator performing the medical imaging procedure.

One advantage resides in providing standardized imaging protocols.

Another advantage resides in updating imaging protocols based on information from a current imaging examination.

Another advantage resides in providing automated or semiautomated adjustment of an imaging protocol based in part on information obtained from a radiology operations command center (ROCC) system for establishing a communication pathway between an expert technician and a technician performing an imaging exam so that the expert technician can assist the technician performing the imaging exam.

Another advantage resides in homogenizing display screens used in imaging protocols using imaging devices from different vendors.

Another advantage resides in detecting, proposing, and/or enforcing adherence to imaging protocols.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows an illustrative apparatus for providing remote assistance in accordance with the present disclosure.

FIG. 2 shows an example flow chart of operations suitably performed by the apparatus of FIG. 1.

FIG. 3 shows a schematic of a server of the apparatus of FIG. 1.

### DETAILED DESCRIPTION

Historically, updates to the imaging protocols of a hospital are typically done manually and in an ad hoc manner. The following discloses an automated system to search relevant medical literature and the Internet to automatically extract suggested updates to the imaging protocols repository of the hospital, and to provide contextually driven imaging protocol update suggestions to the imaging operator and/or a remote expert based on an automatically determined context of a current imaging examination. The extracted suggested updates may also be presented to a radiology department administrator for consideration when updating the standard imaging protocols of the radiology department. To this end, natural language processing (NLP) such as keyword searching on imaging modality, scan protocol names and settings (e.g. SSFP, TE, TR, FOV, et cetera for MRI), names of prominent researchers and/or medical institutions, names of diseases or other clinical condition indicators, and so forth. Since different imaging device vendors sometimes use different names for the same imaging protocol and/or parameters, the NLP includes mapping of these terms to the target imaging device. Dates of publication are also extracted. The extracted information will generally not amount to a complete machine executable protocol, but more typically may include information such as certain suggested scan setting values for a particular disease, as an example. This creates a protocol update suggestions repository indexed by publication date, source, disease, and imaging modality and protocol. Each protocol update suggestion also includes a hyperlink to the source publication.

During an imaging examination, the protocol update suggestions repository is searched based on information on the imaging examination extracted by the control display/console mirroring component of a Remote Operations Command Center (ROCC), or extracted from other sources such as an exam card, radiology schedule, patient record, or the like. If a relevant protocol update suggestion is identified by this search, it is presented, e.g. on a pop-up window of the imaging device controller graphical user interface (GUI). The pop-up also includes a hyperlink to the source publication. In another use case, the protocol update suggestion may be provided on the GUI of a radiology workstation as a radiologist is preparing the imaging protocol for an upcoming imaging examination. In another case, the protocol update suggestion may be provided to a central unit or board of the institution to investigate, or adopt it in a centralized way per institution. Optionally, the local operator can select to adopt the suggestion in which case the update is automatically populated into the patient's scan setup or protocol.

The following also discloses a system that employs control display/console mirroring of the imaging device controller during an imaging examination to identify the imaging protocol being employed and to detect any deviations from that imaging protocol. For example, You Only Look Once (YOLO) or another object detector can analyze scout images acquired during scan setup to identify the target anatomy, and the scan settings (or protocol name itself, if displayed) can be extracted from the mirrored controller display by optical character recognition (OCR), optionally in view of supplemental information such as a priori known GUI dialog screens of the imaging device controller, to identify both the protocol and the scan settings being used. This information is compared against the default scan settings for that protocol and any deviations can be identified. Deviations can be identified on the scanner controller GUI, and could also be logged for use in monitoring to identify frequent deviations from the standard protocol. This latter information could be fed back to the protocol update suggestions repository of the first disclosure as suggested modifications for future imaging examinations. Tracking protocol deviations automatically can be useful for quality monitoring and assurance across techs, scanners and imaging sites to ensure intra-institutional consistency.

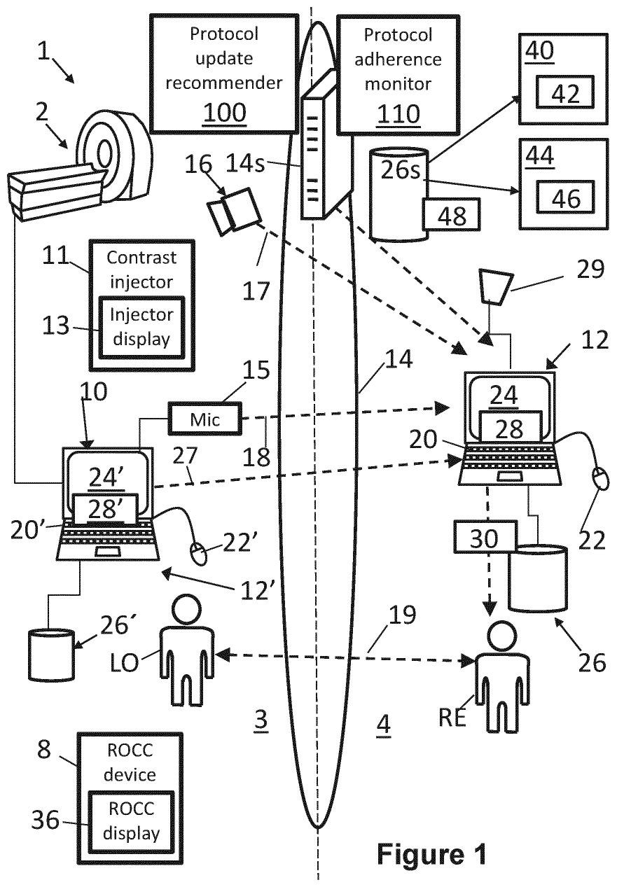

With reference to FIG. 1, an ROCC apparatus 1 for providing assistance from a remote expert RE (or e.g., a senior imaging technologist, radiologist, or so forth; more generally, a remote user RE) to a local operator LO is shown. As shown in FIG. 1, the local operator LO, who operates a medical imaging device (also referred to as an image acquisition device, imaging device, and so forth) 2, is located in a medical imaging device bay 3, and the remote expert RE is disposed in a remote service location or center 4. It should be noted that the remote expert RE may not necessarily directly operate the medical imaging device 2, but rather provides assistance to the local operator LO in the form of advice, guidance, instructions, or the like. The remote location 4 can be a remote service center, a radiologist's office, a radiology department, and so forth. The remote location 4 may be in the same building as the medical imaging device bay 3 (this may, for example, in the case of a remote expert RE who is a radiologist tasked with peri-examination image review), but more typically the remote service center 4 and the medical imaging device bay 3 are in different buildings, and indeed may be located in different cities, different countries, and/or different continents. In general, the remote location 4 is remote from the imaging device bay 3 in the sense that the remote expert RE cannot directly visually observe the imaging device 2 in the imaging device bay 3 (hence optionally providing a video stream as described further herein).

The image acquisition device 2 can be a Magnetic Resonance (MR) image acquisition device, a Computed Tomography (CT) image acquisition device; a positron emission tomography (PET) image acquisition device; a single photon emission computed tomography (SPECT) image acquisition device; an X-ray image acquisition device; an ultrasound (US) image acquisition device; or a medical imaging device of another modality. The imaging device 2 may also be a hybrid imaging device such as a PET/CT or SPECT/CT imaging system. While a single image acquisition device 2 is shown by way of illustration in FIG. 1, more typically a medical imaging laboratory will have multiple image acquisition devices, which may be of the same and/or different imaging modalities. For example, if a hospital performs many CT imaging examinations and relatively fewer MRI examinations and still fewer PET examinations, then the hospital's imaging laboratory (sometimes called the “radiology lab” or some other similar nomenclature) may have three CT scanners, two MRI scanners, and only a single PET scanner. This is merely an example. Moreover, the remote service center 4 may provide service to multiple hospitals and other types of imaging sites. The local operator controls the medical imaging device 2 via an imaging device controller 10. The remote expert is stationed at a remote electronic processing device 12 (or, more generally, an electronic controller 12).

Some types of imaging modalities and some types of imaging examinations may employ a contrast agent. For example, some types of MRI angiography imaging examinations employ a gadolinium-based magnetic contrast agent to observe blood flow into and out of an anatomical organ or region. To provide for such contrast-enhanced imaging, a programmable contrast injector 11 with a display 13 is configured to inject the patient with a contrast agent. In such an embodiment, the injector display 13 may be monitored by a further ROCC sensor such as a camera viewing the injector display 13.

As used herein, the term “medical imaging device bay” (and variants thereof) refer to a room containing the medical imaging device 2 and also any adjacent control room containing the medical imaging device controller 10 for controlling the medical imaging device. For example, in reference to an MRI device, the medical imaging device bay 3 can include the radiofrequency (RF) shielded room containing the MRI device 2, as well as an adjacent control room housing the medical imaging device controller 10, as understood in the art of MRI devices and procedures. On the other hand, for other imaging modalities such as CT, the imaging device controller 10 may be located in the same room as the imaging device 2, so that there is no adjacent control room and the medical bay 3 is only the room containing the medical imaging device 2. In addition, while FIG. 1 shows a single medical imaging device bay 3, it will be appreciated that the remote service center 4 (and more particularly the remote electronic processing device 12) is in communication with multiple medical bays via a communication link 14, which typically comprises the Internet augmented by local area networks at the remote expert RE and local operator LO ends for electronic data communications. In addition, while FIG. 1 shows a single remote service center 4, it will be appreciated that the medical imaging device bays 3 is in communication with multiple medical bays via the communication link 14.

As diagrammatically shown in FIG. 1, in some embodiments, a camera 16 (e.g., a video camera) is arranged to acquire a video stream 17 of a portion of a workspace of the medical imaging device bay 3 that includes at least the area of the imaging device 2 where the local operator LO interacts with the patient, and optionally may further include the medical imaging device controller 10. In other embodiments, a microphone 15 is arranged to acquire an audio stream 18 of the workspace that includes audio noises occurring within the medical imaging device bay 3 (e.g., verbal instructions by the local operator LO, questions from the patient, and so forth). The video stream 17 and/or the audio stream 18 is sent to the remote electronic processing device 12 via the communication link 14, e.g., as a video stream received via a secure Internet link.

The communication link 14 also provides a natural language communication pathway 19 for verbal and/or textual communication between the local operator and the remote operator. For example, the natural language communication link 19 may be a Voice-Over-Internet-Protocol (VOIP) telephonic connection, an online video chat link, a computerized instant messaging service, or so forth. Alternatively, the natural language communication pathway 19 may be provided by a dedicated communication link that is separate from the communication link 14 providing the data communications 17, 18, e.g., the natural language communication pathway 19 may be provided via a landline telephone. In some embodiments, the natural language communication link 19 allows a local operator LO to call a selected remote expert RE. The call, as used herein, can refer to an audio call (e.g., a telephone call), a video call (e.g., a Skype or Facetime or other screen-sharing program), or an audio-video call. In another example, the natural language communication pathway 19 may be provided via an ROCC device 8 with a display device 36. The ROCC Device 8 is for example a tablet, mobile phone, desktop PC, workstation, etc. For example, an “app” can run on the ROCC device 8 (operable by the local operator LO) and the remote electronic processing device 12 (operable by the remote expert RE) to allow communication (e.g., audio chats, video chats, and so forth) between the local operator and the remote expert.

FIG. 1 also shows, in the remote service center 4 including the remote electronic processing device 12, such as a workstation, a workstation computer, or more generally a computer, which is operatively connected to receive and present the video stream 17 of the medical imaging device bay 3 from the camera 16 and/or to the audio stream 18. Additionally or alternatively, the remote workstation 12 can be embodied as a server computer or a plurality of server computers, e.g., interconnected to form a server cluster, cloud computing resource, or so forth. The workstation 12 includes typical components, such as an electronic processor 20 (e.g., a microprocessor), at least one user input device (e.g., a mouse, a keyboard, a trackball, and/or the like) 22, and at least one display device 24 (e.g., an LCD display, plasma display, cathode ray tube display, and/or so forth). In some embodiments, the display device 24 can be a separate component from the remote electronic processing device 12. The display device 24 may also comprise two or more display devices. The electronic processor 20 is operatively connected with a one or more non-transitory storage media 26. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the workstation 12, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a graphical user interface (GUI) 28 for display on the remote operator display device 24. The video stream 17 from the camera 16 can also be displayed on the display device 24, and the audio stream 18 can be output on the remote electronic processing device 12 via a loudspeaker 29. In some examples, the audio stream 18 can be an audio component of an audio/video stream (such as, for example, recording as a video cassette recorder (VCR) device would operate).

FIG. 1 shows an illustrative local operator LO, and an illustrative remote expert RE. However, in a Radiology Operations Command Center (ROCC) as contemplated herein, the ROCC provides a staff of remote experts who are available to assist local operators LO at different hospitals, radiology labs, or the like. Each remote expert RE can operate a corresponding remote electronic processing device 12. The ROCC may be housed in a single physical location or may be geographically distributed. For example, in one contemplated implementation, the remote expert RE are recruited from across the United States and/or internationally in order to provide a staff of remote experts with a wide range of expertise in various imaging modalities and in various imaging procedures targeting various imaged anatomies. A server computer 14s can be in communication with the medical imaging bay 3 and the remote service center 4 with one or more non-transitory storage media 26s. The non-transitory storage media 26s may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the server computer 14s, various combinations thereof, or so forth. It is to be understood that any reference to a non-transitory medium or media 26s herein is to be broadly construed as encompassing a single medium or multiple media of the same or different types. Likewise, the server computer 14s may be embodied as a single electronic processor or as two or more electronic processors. The non-transitory storage media 26s stores instructions executable by the server computer 14s.

The medical imaging device controller 10 in the medical imaging device bay 3 also includes similar components as the remote electronic processing device 12 disposed in the remote service center 4. Except as otherwise indicated herein, features of the medical imaging device controller 10, which includes a local workstation 12′, disposed in the medical imaging device bay 3 similar to those of the remote workstation 12 disposed in the remote service center 4 have a common reference number followed by a “prime” symbol, and the description of the components of the medical imaging device controller 10 will not be repeated. In particular, the medical imaging device controller 10 is configured to display a GUI 28′ on a display device or controller display 24′ that presents information pertaining to the control of the medical imaging device 2, such as configuration displays for adjusting configuration settings an alert 30 perceptible at the remote location when the status information on the medical imaging examination satisfies an alert criterion of the imaging device 2, imaging acquisition monitoring information, presentation of acquired medical images, and so forth. A screen mirroring data stream 27 carries the content presented on the display device 24′ of the medical imaging device controller 10. The screen mirroring data stream 27 is produced by a screen mirroring connection configured to perform the screen mirroring of the display 24′ of the imaging device controller 10. The screen mirroring data stream 27 can be acquired in various ways. In one approach, the screen mirroring data stream 27 can be acquired by a screen mirroring connection comprising screen mirroring software running on the imaging device controller 10. In another approach, the control display/console mirroring is obtained by a screen mirroring connection comprising screen scraping hardware interposed between the display device 24′ of the imaging device controller 10 and the at least one electronic processor 20′ (e.g., video card or the like) that outputs the video signal. In this latter approach, the screen scraping hardware may, by way of nonlimiting illustrative example, include a video splitter that splits the video signal, with one video signal being sent to the display device 24′ and the other being sent to the remote electronic processing device 12 as the screen mirroring data stream 27. The communication link 14 allows for screen sharing between the display device 24 in the remote service center 4 and the display device 24′ in the medical imaging device bay 3. The GUI 28′ includes one or more dialog screens, including, for example, an examination/scan selection dialog screen, a scan settings dialog screen, an acquisition monitoring dialog screen, among others. The GUI 28′ can be included in the video stream 17 and displayed on the remote workstation display 24 at the remote location 4.

Hence, the ROCC apparatus 1 provides the remote expert RE with remote monitoring of the medical imaging examination being conducted by the local operator LO. The remote monitoring includes, for example, the video stream 17 from the camera 16, the audio stream 18, and the screen mirroring data stream 27.

The server 14s also stores, in the non-transitory storage media 26s, an imaging protocols repository 40 of a medical facility that stores a plurality of imaging protocols 42 to be used in medical imaging examinations. In some examples, the imaging protocols repository 40 includes imaging protocols 42 for a plurality of medical facilities. In addition, the server 14s also stores a protocol update suggestions repository 44 that stores suggested updates 46 to the imaging protocols 42. In addition, the server 14s stores medical literature 48 including at least one of proprietary subscription medical literature and/or medical literature retrieved from the Internet.

Furthermore, as disclosed herein, the server 14s performs a protocol update recommendation method or process 100 for extracting protocol update recommendations from the medical literature and/or the Internet and providing in-examination update recommendations based on the exam context determined from the ROCC monitoring (e.g., analysis of the video stream 17 and screen mirroring data stream 27). The protocol update recommender 100 thus advantageously leverages the ROCC infrastructure for the secondary purpose of automatically providing imaging protocol update recommendations.

As also disclosed herein, the server 14s may also implement a protocol adherence monitor 110. This component again leverages exam context determined from the ROCC monitoring (e.g., analysis of the video stream 17 and screen mirroring data stream 27) to identify the imaging protocol being used and to detect any deviations from that protocol. Alerts generated by the protocol adherence monitor 110 may be sent to the local operator LO via the medical imaging device controller 10, the ROCC device 8, or another electronic device accessible in the medical imaging device bay 3.

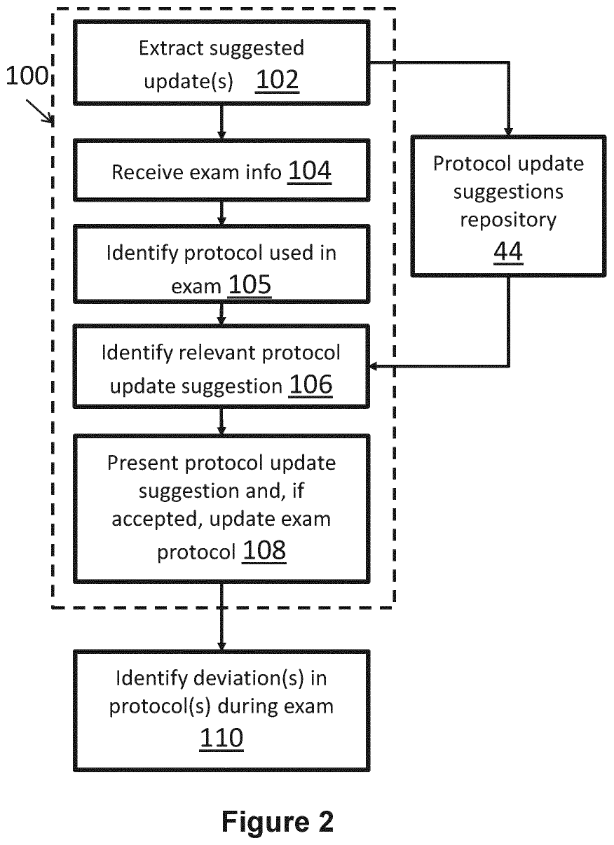

With reference to FIG. 2, and with continuing reference to FIG. 1, an illustrative embodiment of the protocol update recommender method 100 is diagrammatically shown as a flowchart. To begin the update recommender method 100, at an operation 102, the medical literature 48 is processed to extract suggested updates 46 to the imaging protocols repository 40, and adding the suggested updates 46 to the protocol update suggestions repository 44. This operation 102 is typically performed outside of the context of the current imaging examination, and indeed may be a continuous process of mining the medical literature 48 to keep the imaging protocols repository 40 up to date with the latest protocol developments reported in the medical literature.

To extract the suggested updates 46 in the operation 102, the server 14s is configured to apply a natural language process (NLP) to the medical literature 48 to extract metadata associated with each suggested update 46. The metadata can include, for example, one or more of an imaging modality, a scan protocol name, one or more author names, one or more author medical institutions, a disease for which the suggested update is applicable, a clinical condition indicator for which the suggested update is applicable, and a publication date. It will be appreciated that the processing of the medical literature 48 can be performed prior to, or during, a medical imaging procedure.

At an operation 104, during a medical imaging procedure, information related to a current medical imaging examination of the patient is received at the server computer 14s. The received information can include a variety of different types of information. For example, the received information can include information obtained from remote monitoring, e.g., by analyzing the video stream 17, audio stream 18, and/or the screen sharing video stream 27. The received information can also include, for example, inputs related to the medical imaging examination that are received from the remote monitoring by the ROCC and/or input by the local operator LO (i.e., the local imaging operator LO) via the ROCC device 8, information from prior studies or medical imaging examinations, reimbursement guidelines for medical imaging examinations; inputs related to the medical imaging examination that are input by a radiologist (for example, as annotations to the examination order made by the radiologist) and/or the remote expert RE via the remote electronic processing device 12; and/or so forth. The received information is then transmitted to the remote electronic processing device 12 for analysis by the remote expert RE.

At an operation 105, the imaging protocol being employed in the imaging examination (or a current imaging protocol of a current step of the examination) is identified based on the information related to the current medical imaging examination collected in the operation 104. This may be done in various ways, such as directly reading the protocol name from the GUI display captured in the screen sharing video screen 27 using OCR, or matching user dialogs captured in the screen sharing video screen 27 with the imaging protocol, or by analyzing scout images contained in the screen sharing video screen 27 to identify the imaged anatomy, various combinations thereof, and/or so forth.

At an operation 106, the protocol update suggestions repository 44 is accessed to determine whether it contains any protocol update suggestions for the imaging protocol identified in the operation 105. If so, then at an operation 108 the protocol update suggestion retrieved from the protocol update suggestions repository 44 is presented to the local operator LO on the controller display, ROCC device 8, or the like; and/or is presented to the remote expert RE via the workstation 12. If the presented protocol update suggestion is accepted then the imaging protocol for the current imaging examination can be automatically updated accordingly, or in a variant embodiment the local operator LO manually implements the protocol update suggestion if he or she determines to accept it. To assist in the decision of whether to accept the imaging protocol update suggestion, the hyperlink to the source medical literature (e.g. the specific article from which the suggestion was extracted) may be provided so that it can be conveniently retrieved and reviewed. While the operations 106 and 108 are described for a single update suggestion, these operations could identify two or more imaging protocol update suggestions each of which may be accepted or rejected by the local operator LO. Likewise, if the imaging examination includes multiple imaging protocols (e.g. with/without contrast agent), then the operations 105, 106, and 108 can be repeated for each successive imaging protocol of the imaging examination. One or more of the imaging protocols 42 can thus be updated with the extracted suggested updates 46 from the medical literature 48. To do so, during the medical procedure, one or more of the imaging protocols 42 being used in the medical examination can be identified in the operations 104 and 105. The extracted suggested updates 46 can be retrieved from the protocol update suggestions repository 44 for the identified medical imaging protocol(s) 42 in the operation 106, and presented to the local operator in operation 108.

At the operation 108, the retrieved protocol update suggestion(s) 46 can be displayed on a display device observable by an operator performing the medical imaging procedure (i.e., on the display device 36 of the ROCC device 8 or the display device 24 of the remote electronic processing device 12). The user (i.e., the local operator LO and/or the remote expert RE) can provide a user input via the ROCC device 8 and/or the remote electronic processing device 12 to accept the update suggestion(s) 46, and in response, update the medical imaging procedure to employ the identified medical imaging protocol 42 with the update suggestion 46.

In some examples, a repository update proposal comprising the suggested update(s) 46 from the protocol update suggestions repository 44 and a corresponding medical imaging protocol 42 from the imaging protocols repository 40 can be displayed on the display device 36 of the ROCC device 8 or the display device 24 of the remote electronic processing device 12. The user (i.e., the local operator LO and/or the remote expert RE) can provide a user input via the ROCC device 8 and/or the remote electronic processing device 12 to accept the repository update proposal, and in response to receiving the user input, updating the medical imaging protocol(s) 42 of the repository update proposal with the suggested update(s) 46. To do so, the extracted suggested updates 46 are then mapped to settings of medical imaging protocols 42 to update the imaging protocols 42. In some examples, the suggested updates 46 can be annotated with hyperlinks to the medical literature 48 from which the suggested updates 46 were extracted. The updated imaging protocols 42 can then be indexed, by, for example, one or more of publication date, source, disease, modality, and protocol.

With continuing reference to FIG. 2, after the imaging protocol has been finalized (with or without any updates produced by the protocol update recommender 100), the protocol adherence monitor 110 can be employed during the execution of the imaging examination. As shown in FIG. 2, at the operation 110, in some embodiments, the received examination monitoring information (from the operation 104) and the identified imaging protocol(s) 42 (from the operation 105) can be compared to each other to detect one or more deviations from the identified imaging protocol(s) 42. For example, the identifying of the imaging protocol(s) 42 can include, for example, identifying scan settings and an anatomy being imaged during the medical imaging procedure. The identifying process can be performed using an object detector process. An indication of the detected one or more deviations can be displayed on the display device 36 of the ROCC device 8 or the display device 24 of the remote electronic processing device 12. In some examples, the identified imaging protocol(s) 42 can be updated with the detected one or more deviations.

Furthermore, during the imaging examination, the ROCC infrastructure can enable the remote expert RE to assist the local operator LO. To this end, the natural communication pathway 19 is provided between the local operator LO and the remote expert RE. This may occur in response to the local operator LO making a request for remote expert assistance via the ROCC device 8, and the communication pathway may, by way of nonlimiting illustrative example, comprise a telephonic or videoconference link established between the local operator LO and the assisting remote expert RE, along with sharing of the controller display and imaging bay sensor data with the remote expert RE. Advantageously, however, such involvement of the remote expert RE can be reduced or (in some cases) eliminated entirely by way of automated analysis of the remote monitoring 17, 18, 27 of the medical imaging examination acquired by the ROCC device 8, thus enabling the ROCC device 8 to provide such assistance in an automated fashion without involvement of the remote expert RE thereby freeing the remote expert RE to handle more complex assistive tasks that cannot be performed in such automated fashion.

As disclosed herein, however, the ROCC framework is also used in the protocol update recommender method 100 and the protocol adherence monitor 110 to provide the additional functionality of protocol update suggestions and alerts of protocol deviations during the imaging examination, respectively. Since there may be dozens, hundreds, or more local operators performing imaging examinations under ROCC monitoring at any given time, the method 100 promotes targeted local operator assistance by identifying local operators that are likely to be in a position to assist one another. Notably, the protocol adherence monitor 110 can automatically detect protocol deviations that could adversely affect the imaging examination and notify the local operator LO and/or the remote expert RE of such deviations, thus reducing the stress and workload on both parties, especially the remote expert RE who may be monitoring multiple examinations at any given time.

### EXAMPLES

The following are merely further nonlimiting illustrative examples. The ROCC apparatus 1 includes add-on software feature with no additional hardware required. Using a web crawler in combination with natural language processing, the ROCC apparatus 1 would automatically source new imaging protocol recommendations and clinical guidelines from the scientific literature and major medical and disease associations and add those to a repository. If enabled, the ROCC apparatus 1 would alert the user, typically a radiologist or radiology department head who oversees determining the institution's imaging protocols, to new available protocol updates or clinical guidelines and provide the reference(s) used. Users can also enable alerts for specific conditions, disorders, and diseases that may be of particular interest to their department/facility. The user can then decide if changes should be made to their existing protocols and do so manually (possibly with guidance from a remote expert).

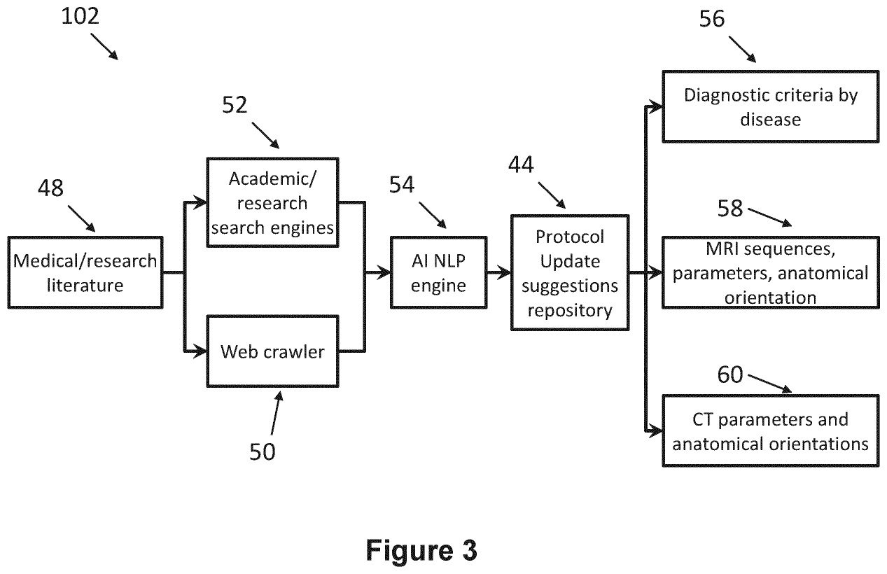

FIG. 3 shows a schematic example of the operation 102 of FIG. 1 according to a more detailed embodiment. The imaging protocols update suggestions repository 44 is a collection of CT/MRI protocol update suggestions for the imaging protocols repository 40 of the institution. The ROCC apparatus 1 can further comprise a web crawler bot 50 incorporating a natural language processing engine 54 that has access to academic/research search engines 52, where it periodically combs through medical and research literature for publications with key terms including but not limited to “CT protocol recommendation”, “updated MRI criteria”, “clinical radiology indications/guidelines”, and “MRI sequence recommendations”. The ROCC apparatus 1 can also keep track of new publications by major research groups or organizations that specialize in disease and/or translational imaging research such as North American Imaging in Multiple Sclerosis Cooperative (NAIMS) and Athinoula A. Martinos Center for Biomedical Imaging, as well as reputable online educational sites and resources run by expert technologists and radiologists that publish recommended protocol parameters such as Oregon Health and Science University, CTisus, Lifespan, MRIMaster. com, Radiopaedia, and MRI Online.

Diagnostic criteria and clinical practices (shown in FIG. 3 with reference character 56) are constantly changing to keep up with new findings, and these often involve the radiology department. For example, the 2021 recommendations on the use of MRI in patients with multiple sclerosis include the use of gadolinium-based contrast agents for specific purposes and “the use of MRI in patients with MS in childhood, during pregnancy, and in the post-partum period” (see, e.g., Wattjes, M. P., Ciccarelli, O., Reich, D. S., Banwell, B., de Stefano, N., Enzinger, C., et al. (2021) 2021 MAGNIMS-CMSC-NAIMS consensus recommendations on the use of MRI in patients with multiple sclerosis. Lancel Neurol, 20(8): 653-670. Doi: 10.1016/S 1474-4422(21)00095-8). Alerting radiologists to these updated recommendations can help to better inform their clinical decisions, ultimately improving the quality of care that patients receive. For CT, intravenous iodinated contrast may be recommended for clinical situations such as evaluation of pyrexia of unknown origin or acute infections (see, e.g., Bhalla, A. S., Das, A., Naranje, P., Irodi, A., Raj, V., & Goyal, A. (2019). Imaging protocols for CT chest: A recommendation. Indian J Radiol Imaging, 29(3): 236-46. Doi: 10.4103/ijri.IJRI_34_19). Furthermore, CT scans are now being recommended for cancer screening practices, especially for lung and colorectal cancer (see, e.g., National Cancer Institute at the National Institutes of Health. (2019). Computed Tomography (CT) Scans and Cancer Fact Sheet—NCI. Retrieved July 7, 2022.). The ROCC apparatus 1 would enable these guideline changes to be brought to the user's attention without requiring extensive manual literature searches on the user's part.

Over the past decade, the American College of Radiology (ACR) has released evolving versions of standardized systems of disease reporting, such as cancer risk. ACR regularly publishes updates to PI-RADS, BI-RADS, LI-RADS and many others. These RADS (Reporting and Data Systems) typically contain guidelines for imaging protocols. The suggested value of certain sequences can be patient-specific and change over time as better sequences are developed or more information is uncovered about the biological mechanisms behind the disease. For example, in PI-RADS, dynamic contrast enhanced (DCE) imaging is not routinely recommended, but DCE is used to judge between clinically significant cancer, likely to require treatment, and less aggressive disease, which could be monitored via active surveillance. Patient information from prior procedures and/or imaging studies combined with latest ACR recommendations can guide appropriate protocol selection.

To reconcile vendor differences in MRI sequence names (shown in FIG. 3 with reference character 58) when adding protocol update suggestions to the protocol update suggestions repository 44, the ROCC apparatus 1 suitably also contains a library of such names and acronyms from the most commonly used vendors around the world. However, new recommendations for MRI sequences are now more commonly made by listing vendor-agnostic parameter values for flip angle (FA), repetition time (TR), echo time (TE), gradient-echo or spin-echo sequence, acquisition matrix, slice thickness and gap, plane, field of view (FOV), contrast bolus timing, anatomical orientation and more such that changes can be made to protocols regardless of which vendor is used. The web crawler 50 sources these new protocol recommendations, extracts all relevant information, and via in the operation 108 (see FIG. 2) summarizes the recommendations on the ROCC device 8 or scanner console screen for the user's convenience. In the illustrative example of FIG. 2, the protocol updates extracted in the operation 102 are treated as protocol update suggestions, with the local operator LO deciding whether to adopt a suggestion in a given imaging examination. Similarly, at a departmental level, a radiology department administrator may decide whether to update the institution's imaging protocols repository 40 with the protocol update suggestions. In one approach for such department updating, systematic side-by-side comparisons with new recommended changes are presented, and are integrated into the institution's imaging protocols repository 40 upon the user's approval. In a variant embodiment, such updates to the institution's imaging protocols repository 40 could be done automatically without human review.

Some further examples of automatic extraction of protocol update suggestions follow.

Radiation dose is to be balanced against image quality when ordering CT scans for patients. Some clinical situations render CT scans that are inappropriate or not ideal, including patients who require repeat imaging or who have contraindications to iodinated contrast. Recent advancements in imaging technology enable the use of low-dose protocols for various clinical purposes including cancer screening. Recommendations for optimizing image quality at lower radiation doses have been published in the literature, and the ROCC apparatus 1 would summarize these changes on the ROCC device 8 or on the console screen for the user's convenience. Information related to CT parameters and anatomical orientations can be output to the user (shown in FIG. 3 with reference character 60). For example, a protocol 42 can be updated with an adapted CT trauma protocol that adjusts the parameters used for different anatomical parts being examined (head, lung, body, bone) including bolus injection protocols to reduce radiation exposure by more than 40% while maintaining good qualitative and quantitative image quality (see, e.g., Kahn, J., Kaul, D., Boning, G., Rotzinger, R., Freyhardt, P., Schwabe, P., . . . & Streitparth, F. (2017). Quality and Dose Optimized CT Trauma Protocol—Recommendation from a University Level-I Trauma Center. Fortscr Rontgenstr, 189:844-54. Doi: 10.1055/s-0043-10899).

In some further examples, an image with the updated protocol(s) 42 can be displayed to the user on the ROCC device 8. In addition to this channel, the information could also be provided via a subscription-based service (e.g. news feeds, mailings, dashboards) to specific users, e.g. to the group of employees evaluating, approving and introducing new protocols at a clinical site. This could be coupled to aggregated information about the current and potentially improved workflow efficiency/image quality in targeted/user specific form depending on the respective interest. From the perspective of the local operator LO at the ROCC device 8, the ROCC apparatus 1 summarizes the recommended parameters for a cervical spine scan at 3T, especially for the delineation of intradural nerve rootlets and neural foramina. The user can close the pop-up as needed, and the hyperlink brings the user directly to the journal article source.

The ROCC device 8 can also collect feedback on the performance of the new research-developed imaging protocol update suggestion 46. Such feedback is scored to assign a rating for the protocol update suggestion 46. This feedback can be collected automatically or could be reported by the local operator LO running the protocol 42 with the update suggestion 46 implemented. For instance, time taken for a protocol could be measured without the involvement of the local operator LO, whereas the improvement in the quality of the image could be reported by the radiologist. The rating thus derived from this feedback can further be used to organically rank the protocol update suggestions in the protocol update suggestions repository 44. A high-ranked research-developed protocol can boost the confidence of the user (radiologist, lead technologist or imaging center) in realizing the benefits by updating their current protocol with research-developed protocol update suggestions 46.

In the following, the protocol adherence monitoring 110 is described in more detail. To perform the operation 110, the server 14s can include one or more modules implemented therein, including a module to recommend protocol for an exam, a module to detect the body part being scanned, a module to infer the protocol of the ongoing exam, a module to identify deviations from recommended protocol, and a module to detect progress of an examination. The output of the modules can be used independently. But the combination (which can be sequential in below order or parallel) of the output/feature information allows to create a more robust generation of the resulting information, i.e. based on the knowledge of the prescribed protocol probabilities can be assigned to the detection of scanned body parts, which in turn should correlate with the protocol of the ongoing exam. For example, a request for images related to a neuro-case would most probably entail a brain-(or spine) protocol and brain/spine image. Various machine learning/AI methods support such sequential or parallel combination of features/measures.

The module to recommend a protocol for an exam is responsible to interact with systems such as EMR, RIS and gather information about the current (optionally also past) exam(s) of the respective patient. Based on this information and the quality policies followed by the corresponding imaging center, this module recommends the protocol(s). In another embodiment, this recommendation could be driven by historical data on similar exams. This module could be realized by text mining algorithms (e.g., named entity recognition, sequence modelling, semantic analysis) on collected information, and recommender system based on connections between exams and users to suggest suitable exam protocols with accuracy.

The module to detect the body part being scanned uses a pre-trained model that detects the name of the body part based on the images displayed on the console screen. The base model can be any object detection model such as you-only-live-once (YOLO), YOLOX etc., and it is trained on a collection of medical images and corresponding metadata containing the body part, sequence name etc. In case of detecting glands or small organs being scanned, such as pancreas, prostate or gallbladder, the detection algorithm could also make use of information such as on-site action of geometry setting around or focusing on area in interest in the obtained images, Image segmentation could also be applied. An image displayed on the ROCC device 8 can include, for example, text next to a red box that represents the predicted name of body part and the confidence in that prediction. Since these images appear during the course of a patient scan across all vendors, it enables the estimation of body part being scanned. Note that, this pre-trained model is generally specific to a modality but could be extended to be multi-modal by normalization, mapping and/or parameter space extension. In another embodiment, this module can have a feedback loop where the local operator or the remote expert can state whether the detected scanned body part is correct or not.

The module to infer the protocol of the ongoing exam is responsible to extract information related to the sequences such as sequence names that are completed, sequence names that are to be run, etc. from the console screen. It uses techniques such as YOLO or pattern matching algorithms to identify the region of interest. The text from this region can be extracted using optical character recognition (OCR) algorithms such as text recognition. In case the on-site local operators frequently modify the current protocol by adding or deleting sequences, the module could save several frames per second in buffer to capture these changes and make better inference. The inference of protocol could also consider the detection result from the module to detect the body part being scanned. Besides, detected keywords such as T1, T2, contrast agent name in the sequence names could also be used. The interaction between the body part detection module and the protocol inference module could help improve accuracy of detection on examined body part and ongoing sequences.

The module to identify deviations from recommended protocol receives a list of pre-defined sequences from the recommended protocol from the protocol recommending module. This list of sequence names is verified with the current list of sequences obtained from the protocol inference module. With body part being scanned is detected by the body part detection module, the remote expert is alerted if there are discrepancies between these two lists of sequences on the scanned body part.

The module to detect progress of the examination can, based on the detection of the protocols of the ongoing exam (from the protocol inference module) and a time series of such detection, the temporal order and state (i.e. waiting vs. running) can be detected by changes in the visualization of the respective step. Even though vendor-specific visualizations are used (e.g. underlying progress bar or font weight), the detection of the sequence worklist (from the protocol inference module) and the completed/running state of a step by differential screen captures at moderate (e.g. 1 per second) framerates is possible.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing instructions for assisting a medical imaging examination of a patient, the instructions, when executed by a processor, cause the processor to:
process medical literature to extract suggested updates to an imaging protocols repository of a medical facility and add the suggested updates to a protocol update suggestions repository;
during a medical imaging procedure, identify a medical imaging protocol from the imaging protocols repository being used in the medical imaging procedure;
retrieve a protocol update suggestion for the identified medical imaging protocol from the protocol update suggestions repository; and
display the retrieved protocol update suggestion on a display device observable by an operator performing the medical imaging procedure.

2. The non-transitory computer readable medium of claim 1, wherein the suggested updated are extracted by:
applying a natural language process to medical literature stored in the imaging protocols repository.

3. The non-transitory computer readable medium of claim 1, wherein the the medical literature includes at least one of proprietary subscription medical literature or medical literature retrieved from the Internet.

4. The non-transitory computer readable medium of claim 1, wherein, to process the medical literature, the instructions, when executed by the processor, further cause the processor to:
extract, from the medical literature, metadata associated with each suggested update including one or more of an imaging modality, a scan protocol name, one or more author names, one or more author medical institutions, a disease for which the suggested update is applicable, a clinical condition indicator for which the suggested update is applicable, and a publication date.

5. The non-transitory computer readable medium of claim 4, wherein, to retrieve the protocol update suggestion, the instructions, when executed by the processor, further cause the processor to: (Currently amended)1 index the updated imaging protocols by one or more of publication date, source, disease, modality, and protocol.

6. The non-transitory computer readable medium of claim 1, wherein the the instructions, when executed by the processor, further cause the processor to:
map the extracted suggested updates to settings of medical imaging protocols of the imaging protocols repository.

7. The non-transitory computer readable medium of claim 1, wherein, to add the suggested updates to the protocol update suggestions repository, the instructions, when executed by the processor, further cause the processor to:
annotate the suggested updates in the protocol update suggestions repository with hyperlinks to the medical literature from which the suggested updates were extracted.

8. The non-transitory computer readable medium of claim 1, wherein the instructions, when executed by the processor, further cause the processor to:
receive examination monitoring information related to the medical imaging procedure and transmitting the examination monitoring information to a remote user, the examination monitoring information including at least a screen mirroring data stream of a controller of a medical imaging device used to perform the medical imaging examination of the patient;
wherein identification of the medical imaging protocol being used in the medical imaging procedure is based on the received examination monitoring information.

9. The non-transitory computer readable medium of claim 8, wherein the instructions, when executed by the processor, further cause the processor to:
provide a natural communication pathway between a local operator performing the imaging examination and a remote user monitoring the medical examination.

10. The non-transitory computer readable medium of claim 1, wherein the instructions, when executed by the processor, further cause the processor to:
receive a user input accepting the update suggestion and in response updating the medical imaging procedure to employ the identified medical imaging protocol with the update suggestion.

11. The non-transitory computer readable medium of any one claim 1, wherein the instructions, when executed by the processor, further cause the processor to:
display a repository update proposal comprising a suggested update from the protocol update suggestions repository and a corresponding medical imaging protocol from the imaging protocols repository; and
in response to receiving a user input accepting the repository update proposal, update the medical imaging protocol of the repository update proposal with the suggested update of the repository update proposal.

12. An apparatus for assisting a medical imaging examination of a patient, the apparatus comprising:
a screen mirroring connection configured to perform screen mirroring of a display of a controller of a medical imaging device used to perform the medical imaging examination; and
at least one processor configured to:
receive examination monitoring information related to the medical imaging examination of the patient and transmitting the examination monitoring information to a remote user, the examination monitoring information including at least a screen mirroring data stream of the display of the controller of the medical imaging device;
identify, from the examination monitoring information, an imaging protocol being employed during the medical imaging examination;
compare the examination monitoring information and the identified imaging protocol to detect one or more deviations from the identified imaging protocol; and
display, on a display device, an indication of the detected one or more deviations.

13. The apparatus of claim 12, wherein, to identify the imaging protocol, the at least one processor is further configured to identify scan settings and an anatomy being imaged during the medical imaging procedure.

14. The apparatus of claim 12, wherein identification of the imaging protocol is performed using an object detector process.

15. The apparatus of claim 12, wherein the at least one processor is further configured to:
update the identified imaging protocol with the detected one or more deviations.

16. A method for assisting a medical imaging examination of a patient, the method comprising:
receiving examination monitoring information related to the medical imaging procedure and transmitting the examination monitoring information to a remote user, the examination monitoring information including at least a screen mirroring data stream of a controller of a medical imaging device used to perform the medical imaging examination of the patient;
processing medical literature to extract suggested updates to an imaging protocols repository of a medical facility and adding the suggested updates to a protocol update suggestions repository based on the received examination monitoring information;
during a medical imaging procedure, identifying a medical imaging protocol from the imaging protocols repository being used in the medical imaging procedure;
retrieving a protocol update suggestion for the identified medical imaging protocol from the protocol update suggestions repository; and
displaying the retrieved protocol update suggestion on a display device observable by an operator performing the medical imaging procedure.

17. The method of claim 16, further comprising:
providing a natural communication pathway between a local operator performing the imaging examination and a remote user monitoring the medical examination.

18. The method of claim 16, further comprising:
mapping the extracted suggested updates to settings of medical imaging protocols of the imaging protocols repository.

19. The method of claim 16, further comprising:
annotating the suggested updates in the protocol update suggestions repository with hyperlinks to the medical literature from which the suggested updates were extracted.

20. The method of claim 16, further comprising:
comparing the examination monitoring information and the identified imaging protocol to detect one or more deviations from the identified imaging protocol.
